(19) United States
(12) Patent Application Publication
(10) Pub. No.: US 2026/0279503 A1
(43) Pub. Date: Sep. 17, 2026

(54) REACTION INFLUENCER SYNTHESIS USING IN-CONTEXT LEARNING AND BAYESIAN OPTIMIZATION

(71) Applicant: University of Rochester, Rochester, NY (US)

(72) Inventors: Andrew D. White, Rochester, NY (US); Marc David Porosoff, Rochester, NY (US); Shane Shlomo Michtavy, Rochester, NY (US); Mayk Caldas Ramos, Rochester, NY (US)

(73) Assignee: University of Rochester, Rochester, NY (US)

(21) Appl. No.: 19/469,735

(22) PCT Filed: Apr. 2, 2024
(86) PCT No.: PCT/US2024/022617

§ 371 Date: Sep. 26, 2025

(51) Int. Cl. G16C 20/10; G06N 7/01; G16C 20/30; G16C 20/70

(52) U.S. Cl. CPC G16C 20/10; G06N 7/01; G16C 20/30; G16C 20/70

(57) ABSTRACT

Techniques described herein pertain to using in-context learning and Bayesian optimization for identifying procedure specifications for synthesizing and testing reaction influencers. A procedure that includes a procedure specification and a label such as yield is used to generate input information for a machine learning model. The input information includes a natural language description of the procedure and a query that includes the label and a normal random variable. A natural language description of a reaction influencer and a procedure for synthesizing and testing the reaction influencer is predicted for the input information using the machine learning model. The machine learning model is then used to predict a set of results including a certainty factor for additional example procedures. Bayesian optimization is performed on the additional example procedures using the certainty factors in an acquisition function to select a candidate example procedure for synthesizing a reaction influencer.

### CROSS-REFERENCE TO RELATED APPLICATIONS

The present application is a non-provisional application of and claims the benefit and priority to U.S. Provisional Application No. 63/456,966, filed Apr. 4, 2023, the entire contents of which is incorporated herein by reference for all purposes.

### STATEMENT REGARDING FEDERALLY SPONSORED RESEARCH

This invention was made with government support under GM137966 awarded by the National Institutes of Health and CBET-1751471 awarded by the National Science Foundation. The government has certain rights in the invention.

### BACKGROUND

Reaction influencers, such as catalysts or other suitable materials, can be useful for enhancing chemical reactions. For example, reaction influencers can be used to increase an efficiency of a chemical reaction, increase a speed of a chemical reaction, increase throughput of the chemical reaction, and the like. Synthesizing a reaction influencer can be difficult. For example, there may be many procedures that can produce a reaction influencer, or variations thereof. In some examples, the reaction influencer may be scarce, expensive, difficult to acquire, difficult to characterize, and the like. Selecting a particular procedure for synthesizing the reaction influencer in which the particular procedure has a highest efficiency, a highest throughput, a highest likelihood to yield a desired reaction influencer, or the like can be difficult, impractical, or impossible using existing techniques.

### BRIEF SUMMARY

Techniques disclosed herein pertain to in-context learning and, more particularly, to using in-context learning and Bayesian optimization for identifying procedure specifications for synthesizing reaction influencers based on natural language.

In various embodiments, a computer-implemented method includes accessing a dataset comprising a set of procedures for synthesizing a plurality of reaction influencers; selecting a first procedure from a subset of procedures of the set of procedures, the first procedure comprising a procedure specification and a corresponding label; generating first input information for the first procedure, wherein the first input information comprises a natural language description of the first procedure and a first query, wherein the first query comprises the corresponding label adjusted by a normal random variable; predicting, based at least in part on the first input information and using a machine learning model, first output information for the first query, wherein the first output information comprises a natural language description of a reaction influencer and a procedure for synthesizing and testing the reaction influencer; selecting a plurality of second procedures from the set of procedures; predicting, using the machine learning model based on previously labeled procedures, a set of results for the plurality of second procedures, wherein the set of results comprises a plurality of certainty factors for the set of results; and selecting a candidate procedure from the set of procedures by performing Bayesian optimization on the plurality of second procedures using the plurality of certainty factors in an acquisition function of the Bayesian optimization.

In some embodiments, each procedure of the set of procedures comprises information identifying an reaction influencer of the plurality of reaction influencers and information identifying procedure conditions for synthesizing and testing the reaction influencer, and wherein each procedure of the subset of procedures comprises the information identifying the reaction influencer, the information describing the procedure conditions, and information identifying a label comprising a yield of a reaction influenced by the reaction influencer.

In some embodiments, the plurality of second procedures is selected from the set of procedures using a maximal marginal relevance function.

In some embodiments, performing Bayesian optimization on the plurality of second procedures comprises performing the following steps: (i) adding an intermediate procedure to a labeled dataset, the intermediate procedure identified from a plurality of procedures using the acquisition function; (ii) generating intermediate input information for the machine learning model based one or more intermediate procedures in the labeled dataset; (iii) predicting, using the machine learning model, intermediate output information for the intermediate input information; (iv) selecting a plurality of intermediate procedures from the set of procedures based on the intermediate output information; (v) predicting, using the machine learning model, results for the plurality of intermediate procedures; (vi) selecting, using the acquisition function, the candidate procedure from the plurality of intermediate procedures; and (vii) repeating steps (i)-(vi) a predetermined number of times.

In some embodiments, generating the intermediate input information for the machine learning model based on the one or more intermediate procedures in the labeled dataset comprises selecting the one or more intermediate procedures from the labeled dataset using a maximal marginal relevance function.

In some embodiments, the first input information further comprises a natural language description of a third procedure, the third procedure selected from a dataset of labeled procedures.

In some embodiments, each respective result of the set of results comprises a yield for a reaction influencer associated with a second procedure of the plurality of second procedures and a certainty factor of the plurality of certainty factors.

Some embodiments include a system including one or more processors and one or more computer-readable media storing instructions which, when executed by the one or more processors, cause the system to perform part or all of the operations and/or methods disclosed herein.

Some embodiments include one or more non-transitory computer-readable media storing instructions which, when executed by one or more processors, cause a system to perform part or all of the operations and/or methods disclosed herein.

The techniques described above and below may be implemented in a number of ways and in a number of contexts. Several example implementations and contexts are provided with reference to the following figures, as described below in more detail. However, the following implementations and contexts are but a few of many.

### BRIEF DESCRIPTION OF THE DRAWINGS

A further understanding of the nature and advantages of various embodiments may be realized by reference to the following figures. In the appended figures, similar components or features may have the same reference label. Further, various components of the same type may be distinguished by following the reference label by a dash and a second label that distinguishes among the similar components. If only the first reference label is used in the specification, the description is applicable to any one of the similar components having the same first reference label irrespective of the second reference label.

FIG. 1 is a data flow diagram of data flow for generating an inverse design of a procedure for synthesizing and implementing a reaction influencer using in-context learning in accordance with various embodiments.

FIG. 2 is a data flow diagram of an iterative data flow for selecting a procedure for synthesizing and testing a reaction influencer using in-context learning and Bayesian optimization in accordance with various embodiments.

FIG. 3 is a data flow diagram of a data flow for generating a prompt using maximal marginal relevance (MMR) and in-context learning in accordance with various embodiments.

FIG. 4 is an example of a computing system for identifying procedure specifications for synthesizing and implementing reaction influencers in accordance with various embodiments.

FIG. 5 is a flowchart of a process for generating inverse designs of reaction procedures using in-context learning and Bayesian optimization in accordance with various embodiments.

FIG. 6 is a flowchart of a process for performing Bayesian optimization for identifying a candidate procedure in accordance with various embodiments.

### DETAILED DESCRIPTION

In the following description, for the purposes of explanation, specific details are set forth to provide a thorough understanding of certain embodiments. However, it will be apparent that various embodiments may be practiced without these specific details. The figures and description are not intended to be restrictive. The word “exemplary” is used herein to mean “serving as an example, instance, or illustration.” Any embodiment or design described herein as “exemplary” is not necessarily to be construed as preferred or advantageous over other embodiments or designs.

Designing new reaction influencers, such as catalysts, may be a critical challenge to address climate change and other contemporary problems. For example, about one-third of the global production may rely on catalytic processes to produce value-added chemicals and fuels from fossil-based resources. One challenge may be navigating the complex experimental space to identify materials that can be used to convert carbon-neutral or negative feed stocks into value-added products. Additionally, aqueous solubility often plays a major role in various fields such as drug design, environmental studies, chemical engineering, and the like. However, accurately predicting aqueous solubility remains a challenge. Conventional approaches often involve direct experimentation, which can be time-consuming, expensive, and sometimes inaccurate, especially for large and diverse sets of compounds.

The techniques described herein may overcome this challenge and others by using in-context learning and Bayesian optimization to identify procedures for synthesizing materials such as reaction influencers. Based on representing materials with the natural language, a machine-learning model such as a large language model can be used to predict material properties from the reaction conditions and synthesis procedures described by the natural language. Using the techniques described herein, inverse designs can be predicted, and actionable procedures can be identified. By obtaining prediction uncertainly from the machine-learning model and using in-context learning, materials and procedures can be optimized using Bayesian optimization. Additionally, using the techniques described herein, chemical properties, reaction outcomes, synthesizability, and the like can be accurately predicted, new reaction influencers may be easier, or more efficient, to synthesize, and performance, such as a solubility or yield, associated with materials and reaction influencers may be easier, or more efficient, to predict.

### Overview

Techniques described herein pertain to identifying procedure specifications for synthesizing and implementing reaction influencers based on Bayesian optimization and in-context learning, for example using machine-learning. The in-context learning may be performed with an artificial intelligence model. In some examples, the artificial intelligence model can be or include a machine-learning model, a generative artificial intelligence model, such as one or more versions of GPT provided by OpenAI, a combination thereof, and the like. In-context learning may involve providing a limited number of examples, sometimes iteratively, to the artificial intelligence model to cause the artificial intelligence model to produce a desired output instead of, for example, directly training, fine-tuning, or otherwise modifying the weights and biases of the artificial intelligence model. Bayesian optimization can involve optimizing an objective function, which may be or include an acquisition function, to select a candidate procedure for synthesizing and implementing the reaction influencer. In some examples, the reaction influencer can be or include a catalyst or other suitable reaction influencer and/or enhancer or other material. Predictions of the synthesis procedures of the reaction influencer provided by the artificial intelligence model can involve a solubility of the reaction influencer, a yield of the reaction influencer from a procedure for synthesizing and testing the reaction influencer, an enhancement to a chemical reaction using the reaction influencer, and the like.

In some examples, a chemical reaction can use a known reaction influencer to initiate the chemical reaction, to speed up the chemical reaction, and the like. In other examples, a base structure of the reaction influencer may be known, but remaining portions of the reaction influencer may be unknown or may vary based on the chemical reaction, based on a procedure for synthesizing the reaction influencer, etc. The reaction influencer may be synthesized using one or more different types of procedures, which may exceed 10,000 different procedures that can generate the reaction influencer having at least the base structure. For example, the one or more different types of procedures may be designed or otherwise configured to receive inputs and generate a reaction influencer product having a base chemical structure of C₂ (e.g., ethylene and/or ethane). Varying the one or more different types of procedures may adjust a yield of the reaction influencer, may adjust remaining portions of the reaction influencer attached to the base structure, and the like. Selecting a procedure from the one or more different types of procedures is difficult. For example, and conventionally, each procedure is manually performed to determine results of each procedure. Manually performing over 10,000 experiments to determine parameters or properties of the reaction influencer for one chemical reaction is onerous and impractical.

The artificial intelligence model can be used with the techniques disclosed herein to address the above-identified problems. For example, the artificial intelligence model can be used with the techniques disclosed herein to predict results of the one or more different procedures for synthesizing and implementing the reaction influencer. Additionally, or alternatively, the artificial intelligence model can be used with the techniques disclosed herein to select an optimal procedure to use for synthesizing and implementing the reaction influencer. By using the artificial intelligence model with the techniques disclosed herein to address the above-identified problems, at least the technical field of chemical discovery and synthesis can be improved. For example, using the artificial intelligence model with the techniques disclosed herein can improve a composition and/or yield of the reaction influencer by selecting the optimal procedure for synthesizing and implementing the reaction influencer. The techniques described herein, while described in the context of reaction influencers and procedure for synthesizing reaction influencers, are not intended to be limited to reaction influencers and such corresponding procedures. Rather, the techniques described herein can be used for identifying procedures for synthesizing other materials (e.g., polyethyleneimine/silica-based hybrid adsorbents for carbon capture). Other improvements are possible by using the artificial intelligence model with the techniques disclosed herein.

In some examples, a process that involves artificial intelligence and optimization techniques can be used to identify a reaction procedure, such as an optimal reaction procedure, for generating a reaction influencer. The process can involve using an initial set of reaction procedures, which can include one or more defined reaction procedures that have results associated with the one or more defined reaction procedures. The process can further involve performing an optimization, such as a Bayesian optimization, on at least a subset of the set of reaction procedures to select a reaction procedure. The results of the selected reaction procedure can be used to generate an input prompt or other input information that can be provided to an artificial intelligence model. The artificial intelligence model can generate an inverse design of a reaction procedure based on the input prompt. The inverse design of the reaction procedure can be used to synthesize the reaction influencer or can be used as input to a second iteration of the process to potentially improve a selection or inverse design of the reaction procedure.

### Reaction Influencer Synthesis

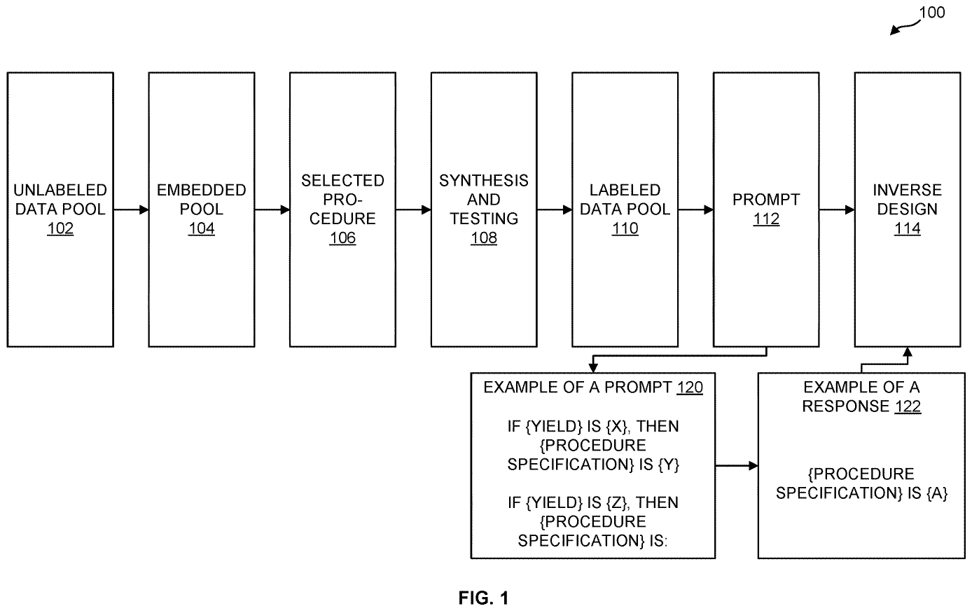

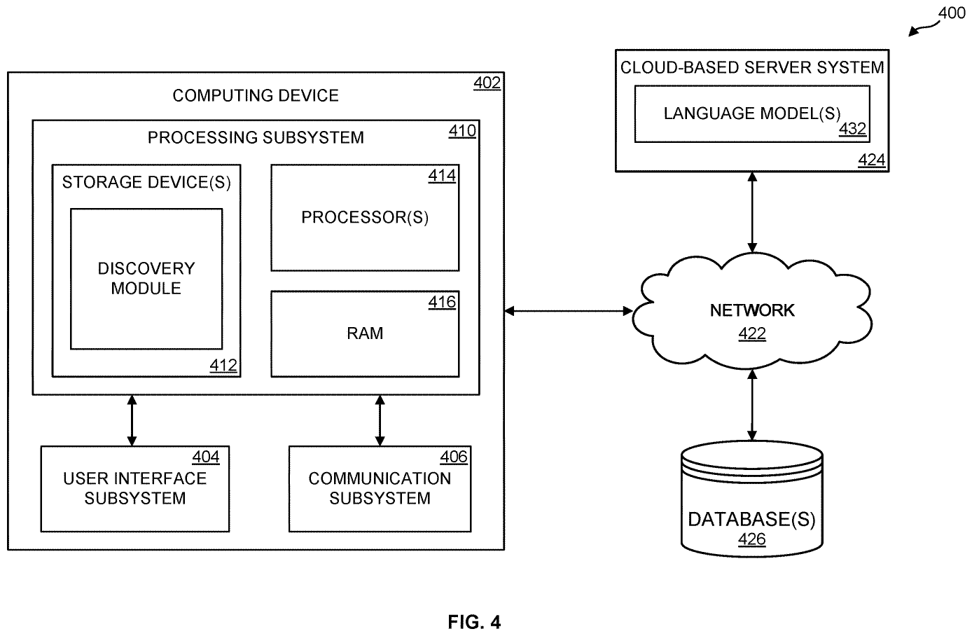

FIG. 1 is a data flow diagram 100 of data flow for generating an inverse design of a procedure for synthesizing a reaction influencer using in-context learning in accordance with various embodiments. As illustrated in FIG. 1, the data flow diagram illustrates one possible, linear example of generating an inverse design of a procedure for synthesizing a reaction influencer using in-context learning. Other suitable examples, including those that are iterative in nature, are possible within the scope of the present disclosure. Additionally, or alternatively, the operations illustrated and/or described with respect to FIG. 1 can be performed by any suitable computing device or computing system such as the computing device 402 or computing system 400 described later with respect to FIG. 4.

In some examples, the data flow diagram 100 may begin with accessing an unlabeled data pool 102. The unlabeled data pool 102 can be stored in a local storage device and/or cloud-based server system and accessed by a processor(s), computing device, computing system, a combination thereof, or the like. The unlabeled data pool 102 can be or include a text-based list or array of data relating to procedures for synthesizing reaction influencers. For example, the unlabeled data pool 102 can be or include a table of examples of procedures for synthesizing a reaction influencer. Each of the examples can be a natural language-based (e.g., text-based) description of the procedure corresponding to the respective example. The procedures for synthesizing the reaction influencer may have previously been performed or may not have previously been performed. For example, one or more procedures for synthesizing the reaction influencer may be theoretical or otherwise planned to be performed but not yet performed such that no results of the one or more experiments are known or available. In some examples, at least a portion of the examples can include results for the procedures associated with the examples. For example, an example in the unlabeled data pool 102 can include a text-based description of a procedure and text-based description of a result or yield (e.g., a material yield) of the procedure. In some examples, examples in the unlabeled data pool can include or be derived from a publicly available dataset.

In some implementations, the unlabeled data pool 102 can be accessed from a database (not shown) and/or cloud-based server system (not shown) that stores datasets for utilization with machine learning models or artificial intelligence models. The unlabeled data pool 102 can include a set of reaction procedures. Each reaction procedure of the set of reaction procedures can include natural language text that identifies a reaction influencer, a set of reaction conditions (e.g., temperature, pressure, flow, etc.), and the like. In some implementations, at least a subset of the set of reaction procedures includes natural language text that identifies the reaction influencer, the set of reaction conditions, and further identifies a yield of a reaction influenced by the reaction influencer (e.g., C₂ yield).

In the case of a reaction involving the oxidative coupling of methane, an example of natural language text for a reaction procedure can include the following: To synthesize Mn—FeMoO₄/SiO₂, SiO₂ (1.0 g) was impregnated with 4.5 mL of an aqueous solution consisting of Mn (0.37 mol), Fe (0.185 mol), and Mo (0.185 mol), at 50° C. for 6 h. Once activated the reaction was run at 900° C. The total flow rate was 15 mL/min (Ar: 10.5 mL/min, CH₄: 3.0 mL/min, O₂: 1.5 mL/min), leading to a contact time of 0.5 s. In another example involving the oxidative coupling of methane, an example of natural language text for a reaction procedure can include the following: To synthesize Mn—FeMoO₄/SiO₂ with a C₂ yield of 4.62, SiO₂ (1.0 g) was impregnated with 4.5 mL of an aqueous solution consisting of Mn (0.37 mol), Fe (0.185 mol), and Mo (0.185 mol), at 50° C. for 6 h. Once activated the reaction was run at 900° C. The total flow rate was 15 mL/min (Ar: 10.5 mL/min, CH₄: 3.0 mL/min, O₂: 1.5 mL/min), leading to a contact time of 0.5 s.

The data flow diagram 100 may proceed to an embedded pool 104 that can be generated by the computing system based at least in part on the unlabeled data pool 102. In some examples, an artificial intelligence model can be used to convert the unlabeled data pool 102 to the embedded pool 104. The artificial intelligence model may be or include a machine-learning model, a generative artificial intelligence model, or the like such as a historical version of GPT provided by OpenAI. The artificial intelligence model can receive the unlabeled data pool 102 as input and can generate, such as via an embedding layer, the embedded pool 104 as an output by performing one or more operations such as vectorization and/or tokenization. In some examples, the artificial intelligence model can convert each reaction procedure of the set of reaction procedures to a vector and/or to a token that can be further processed by the computing system.

The data flow diagram 100 may proceed to a selected procedure 106 that can be selected based on, or otherwise from, the embedded pool 104. In some examples, such as examples in which generating the inverse design is a linear process or examples in which the data flow diagram 100 illustrates an initial step of an iterative process, the selected procedure 106 may be selected at random or pseudo-random. The computing system may randomly, or pseudo-randomly, select a reaction procedure that includes parameters for performing the reaction procedure and the yield or results of the reaction procedure.

The data flow diagram 100 may proceed to synthesis and testing 108, which may involve determining results for the selected procedure 106. In some examples, the results for the selected procedure 106 are known since the selected procedure 106 may be, or may be similar to, a historical procedure that has well-defined results. The results can include a type of structure generated for the reaction influencer, a yield or amount of reaction influencer generated from the selected procedure 106, a solubility of the reaction influencer generated via the selected procedure 106, and the like. In other examples, the results for the selected procedure 106 may not be known and may be determined. The results can be determined by the computing system, or any component or service thereof, by a human or other entity, or the like. The computing system may determine the results by running a simulation of the selected procedure 106. The human or other entity, which may involve using the computing system, may determine the results by manually conducting the selected procedure 106 to determine the actual results. Additionally, or alternatively, the results can be generated by providing an input prompt to the artificial intelligence model to cause the artificial intelligence model to generate a prediction of the results. The results can be provided to the computing system to proceed with the data flow diagram 100.

The data flow diagram 100 may proceed to a labeled data pool 110 that can be generated based at least in part on the synthesis and testing 108. For example, the results determined or otherwise received via the synthesis and testing 108 can be used to generate the labeled data pool 110, which may include a set of labeled data about the selected procedure 106. In examples in which the process represented by the data flow diagram 100 is iterative and on an iterative step after an initial step, the labeled data pool 110 may have pre-existing labeled data about previously processed reaction procedures. The computing system can augment, or generate and/or initialize, the labeled data pool 110 with the results of the selected procedure 106. In some examples, the labeled data pool 110 can include one or more labeled data points corresponding to different reaction procedures. A labeled data point can include a yield or other result of a corresponding reaction procedure, and the labeled data point can include a text representation of the reaction procedure.

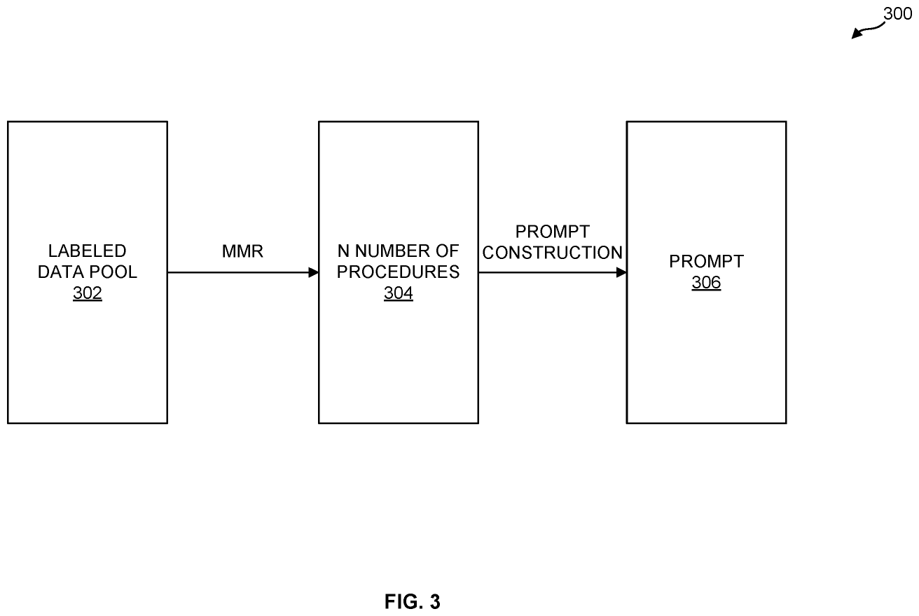

The data flow diagram 100 may proceed to a prompt 112 that can be generated based at least in part on the labeled data pool 110. In some examples, such as examples in which the labeled data pool 110 includes only one labeled data point corresponding to the selected procedure 106, the prompt 112 may be generated based on the selected procedure 106 and/or results of the synthesis and testing 108. In other examples in which more than one labeled data point is included in the labeled data pool 110, a maximal marginal relevance (MMR) operation can be performed on the labeled data pool 110 to select the most relevant data points included in the labeled data pool 110 to use for generating the prompt 112. The prompt 112 may be or include input information configured to be input into the artificial intelligence model to cause the artificial intelligence model to output a particular response, which may be or include a reaction procedure. For example, the prompt 112 may include one or more if-then statements and a question or query regarding randomly perturbed results. Each of the one or more if-then statements can define a reaction procedure and a corresponding result, such as the reaction procedure and the result of the selected procedure 106. The question or query can include a randomly perturbed result followed by an inquiry that inquires as to reaction procedure for yielding the randomly perturbed result. For example, the prompt 112 may ask what the reaction procedure for synthesizing a reaction influencer with a yield multiplied by a random, or pseudo-random, normal value or variable. In some examples, FIG. 3 illustrates one possible example for generating the prompt 112.

Referring now to FIG. 3, which is a data flow diagram 300 of a data flow for generating a prompt using maximal marginal relevance (MMR) and in-context learning in accordance with various embodiments. The data flow diagram 300 may begin with a labeled data pool 302. In some examples, the labeled data pool 302 may be similar or identical to the labeled data pool 110, illustrated and described with respect to FIG. 1. In some examples, the labeled data pool 302 may include a set of labeled data relating to reaction procedures. For example, the labeled data pool 302 includes a set of reaction procedures having a text-based description of a process for performing the corresponding reaction procedure. Additionally, or alternatively, the labeled data pool 302 includes a set of results corresponding to the set of reaction procedures in which each result corresponds to a different reaction procedure and indicates the yield, or other parameters, of the reaction influencer. Additionally, or alternatively, the operations illustrated and/or described with respect to FIG. 3 can be performed by any suitable computing device or computing system such as the computing device 402 or computing system 400 described later with respect to FIG. 4.

A computing system may access or otherwise receive the labeled data pool 302, and the computing system can use the labeled data pool 302 to generate a subset of reaction procedures at N number of procedures 304. For example, the computing system may receive the labeled data pool 302 and perform an MMR operation on the labeled data pool 302 to select the N number of procedures 304. In some examples, MMR combines a search for the most similar examples to a specific sequence, such as the sequences described in each reaction procedure of the set of reaction procedures included in the labeled data pool 302, while maximizing diversity. A similarity of each reaction procedure of the set of reaction procedures with respect to a selected procedure, such as selected procedure 106, which may be selected randomly, pseudo-randomly, or based on Bayesian optimization, etc., can be determined in the MMR operation.

The MMR operation can output the N number of procedures 304 based at least in part on an output of the MMR operation. The N number of procedures 304 can include one reaction procedure, two reaction procedures, three reaction procedures, four reaction procedures, five reaction procedures, or more than five reaction procedures. In some implementations, the N number of procedures 304 can be determined based on a token limit (e.g., 2048 token limit) of the artificial intelligence model. Additionally, or alternatively, each reaction procedure included in the N number of procedures 304 can include a corresponding result such as a yield of reaction influencer. In some examples, N may be adjusted based on a use-case for the procedures included in the N number of procedures 304. For example, N may be selected based on a character limit or word limit for an input to an artificial intelligence model.

The computing system can use the N number of procedures 304 to construct a prompt 306. The prompt 306 may be constructed for input into an artificial intelligence model such as GPT-3, GPT-3.5, GPT-4 (all provided by OpenAI), or any other suitable artificial intelligence model. The prompt 306 may include one or more portions. A first portion of the prompt 306 may include an in-context learning portion. The in-context learning portion of the prompt 306 may provide training data or examples, or pseudo-training data or examples, to the artificial intelligence model in which the artificial intelligence model can learn from. In some examples, the training data or example, or pseudo-training data or example, can include the N number of procedures 304. Since the N number of procedures 304 includes labeled data, such as the results of each of the reaction procedures, then the first portion of the prompt 306 is effectively teaching the artificial intelligence model by providing the training data or example, or pseudo-training data or example. The first portion of the prompt 306 can include one or more if-then statements, though other configurations for providing the training data or example, or pseudo-training data or example, to the artificial intelligence model via the prompt 306 are possible. A second portion of the prompt 306 can include a question or some of an if-then statement. For example, the second portion of the prompt 306 can provide a result adjusted by a random, or pseudo-random, normal value and can ask the artificial intelligence model to provide a reaction procedure for attaining the adjusted result. In other examples, the second portion of the prompt 306 may present an if-statement with the adjusted result and prompt a response from the artificial intelligence model by not including a full then-statement.

The computing system can construct the prompt 306 using at least the first portion and the second portion to ensure that the artificial intelligence model is being provided context for in-context learning and to ensure that a desired response is properly prompted. The prompt 306 may include natural language text and/or other information. The computing system may provide the prompt 306 to the artificial intelligence model, and the computing system may receive a response that includes natural language from the artificial intelligence model that may be understood by an end user of the computing system (e.g., a person).

Referring now back to FIG. 1, the data flow diagram 100 may proceed to an inverse design 114, which can be generated by the artificial intelligence model. For example, the computing system can provide the prompt 112 as input to the artificial intelligence model, and the artificial intelligence model can generate the inverse design 114. The inverse design 114 can be or include a reaction procedure that can be used to synthesize a reaction influencer based on a yield or other result indicated by the prompt 112. In some examples, the inverse design 114 can be used to synthesize the reaction influencer, and, in other examples, the inverse design 114 can be provided as input for the computing system to iterate the process, or a process similar thereto, illustrated by the data flow diagram 100.

In some examples, the inverse design 114 can be generated as an example of a response 122 by the artificial intelligence model using an example of a prompt 120. The example of a prompt 120 may have the structure of: if the yield of the reaction influencer is a first value (X), then the procedure specification for synthesizing the reaction influencer is a first specification (Y); if the yield of the reaction influencer is (Z), then the reaction specification is, and so on. The then-statement that is not complete can serve to prompt the artificial intelligence model to provide the example of the response 122, which may have the structure of: the procedure specification for synthesizing the reaction influencer is a second specification (A). In some examples, the second specification (A) may be a prediction provided by the artificial intelligence model based on the example of the prompt 120.

An example of a prompt can include: If the {C₂ yield} is {4.62}, then the {experimental_procedure} is {To synthesize Mn—FeMoO4/SiO2, SiO2 (1.0 g) was impregnated with 4.5 mL of an aqueous solution consisting of Mn (0.37 mol), Fe (0.185 mol), and Mo (0.185 mol), at 50° C. for 6 h. Once activated the reaction was run at 900° C. The total flow rate was 15 mL/min (Ar: 10.5 mL/min, CH4: 3.0 mL/min, O2: 1.5 mL/min), leading to a contact time of 0.5 s.} . . . Q: If {C₂ yield} is {4.62*np.random.normal(μ,σ)}, then the {experimental_procedure} is:. An example of the response to the prompt can include: A: {To synthesize Mn—Na2WO4/SiCnf, SiCnf (1.0 g) was impregnated with 4.5 mL of an aqueous solution consisting of Mn (0.37 mol), Na (0.37 mol), and W (0.185 mol), at 50° C. for 6 h. Once activated the reaction was run at 900° C. The total flow rate was 20 mL/min (Ar: 8.0 mL/min, CH4: 9.0 mL/min, O2: 3.0 mL/min), leading to a contact time of 0.38 s.}. In some examples, μ and σ can include hyperparameters that can be tuned.

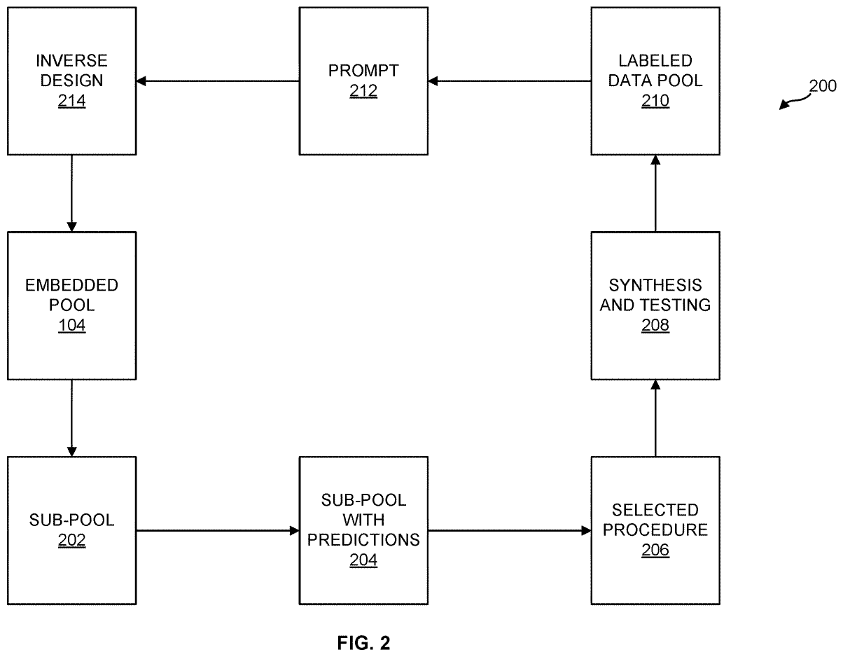

FIG. 2 is a data flow diagram 200 of an iterative data flow for selecting a reaction procedure for synthesizing a reaction influencer using in-context learning and Bayesian optimization in accordance with various embodiments. Operations indicated by the data flow diagram 200 may be similar to the operations indicated by the data flow diagram 100 as illustrated and described with respect to FIG. 1. Additionally, or alternatively, the operations indicated by the data flow diagram 200 may be iterated a suitable number of times to reach a desired outcome. Additionally, or alternatively, the operations illustrated and/or described with respect to FIG. 2 can be performed by any suitable computing device or computing system such as the computing device 402 or computing system 400 described later with respect to FIG. 4.

The data flow diagram 200 may begin with the embedded pool 104. In some examples, the data flow diagram 200 is circular such that “begin” can involve proceeding to another iteration of the process illustrated by the data flow diagram 200. In some examples, the embedded pool 104 may have been generated using an artificial intelligence model or other suitable model that can perform embedding. For example, the artificial intelligence model can receive the unlabeled data pool 102 and can use an embedding layer to convert the data included in the unlabeled data pool 102 to embedded data included in the embedded pool 104.

The data flow diagram 200 may proceed to a sub-pool 202 that can be based on the embedded pool 104. In some examples, the sub-pool 202 may include an N-sized set of reaction procedures from the embedded pool 104. N may be configured by a user or may otherwise be optimized, for example by the computing system. In some examples, N may range from one, two, three, four, or five reaction procedures to 16, 17, 18, 19, or more than 19 reaction procedures. The computing system may select the sub-pool 202 from the embedded pool 104 based at least in part on an input design for a reaction procedure. For example, the computing system may receive an inverse design generated by the artificial intelligence model and may compare the inverse design to the reaction procedures included in the embedded pool 104. In some examples, the computing system may perform an MMR technique using the inverse design to select the N-sized set of reaction procedures from the embedded pool 104 to include in the sub-pool 202.

The data flow diagram 200 may proceed to a sub-pool with predictions 204 based on the sub-pool 202. The sub-pool 202 may be provided to the artificial intelligence model, which can generate predictions about the reaction procedures included in the sub-pool 202. For example, the reaction procedures included in the sub-pool 202 can be used to generate an input prompt to provide to the artificial intelligence model to cause the artificial intelligence model to generate the predictions along with corresponding confidence, or uncertainty, values. In some examples, the input prompt that can be provided to the artificial intelligence model can be or include a specification for carrying out a reaction procedure included in the sub-pool 202 and a query requesting predicted results, such as a yield, based on the specification. The input prompt may include a set of input prompts in which each input prompt of the set of input prompts can relate to a different reaction procedure included in the sub-pool 202. For example, if there are 16 reaction procedures included in the sub-pool 202, then the set of input prompts may include 16 different input prompts corresponding to the reaction procedures included in the sub-pool 202. Additionally, or alternatively, the predictions may include a number of predictions corresponding to the number of reaction procedures included in the sub-pool 202.

In some implementations, an input prompt can be generated for each reaction procedure included in the sub-pool 202. The input prompt generated for a respective procedure can include a natural-language (e.g., text-based) description of the respective procedure and a query. The query can be or correspond to information to be predicted or desired to be predicted from the artificial intelligence model (e.g., a predicted yield or result of the respective procedure). In some implementations, the input prompt may be or include input information configured to be input into the artificial intelligence model to cause the artificial intelligence model to output a particular response, which may be or include a result or yield of a reaction procedure. For example, the prompt may include one or more if-then statements and a question or query. Each of the one or more if-then statements can define a reaction procedure and a corresponding result, such as a reaction procedure and the result of a respective procedure in the labeled pool 302. The question or query can include an inquiry that inquires as to a yield or result for a respective reaction procedure in the sub-pool 202. For example, the input prompt may ask what the yield or result that is synthesized by a respective reaction procedure in the sub-pool 202.

The one or more if-then statements can serve as context for the question or query. In some implementations, the one or more if-then statements can be generated from reaction procedures and their corresponding results in the labeled data pool 302. The reaction procedures and their corresponding results for an input prompt for a given reaction procedure of the sub-pool 202 can be selected from the labeled data pool 302 using the MMR operation. For example, as described above with respect to FIG. 3, the MMR operation can output the N number of procedures based at least in part on an output of the MMR operation. In some implementations, the one or more if-then statements generated from reaction procedures and their corresponding results in the labeled data pool 302 serve as an in-context learning portion and may provide training data or examples, or pseudo-training data or examples, to the artificial intelligence model in which the artificial intelligence model can learn from in predicting the result or yield for a given reaction procedure selected from the sub-pool 202.

The input prompt may include natural language text describing the reaction procedures along with a question or other suitable type of prompt for the artificial intelligence model to answer. In some examples, the predictions may be or include the yield of reaction influencer or other results from the corresponding reaction procedure. The confidence, or uncertainty, values may indicate an uncertainty associated with the corresponding yield or other results of the predictions generated by the artificial intelligence model.

The data flow diagram 200 may proceed to a selected procedure 206, which may be similar or identical to the selected procedure 106 but may be selected using a different technique than the selected procedure 106. For example, instead of being selected randomly, the selected procedure 206 may be selected based on an optimization technique. The optimization technique can be or include Bayesian optimization or other suitable optimization techniques. For example, the computing system can provide a predicted yield and the confidence values for each reaction procedure to a Bayesian optimization service that may incorporate an acquisition function using the yield and the confidence values to determine which reaction procedure included in the sub-pool with predictions 204 is optimal for selecting as the selected procedure 206. In some examples, the acquisition function can be or include an expected improvement acquisition function, an upper confidence bound acquisition function, other suitable acquisition functions, or any combination thereof.

The data flow diagram 200 may proceed to synthesis and testing 208. In some examples, the selected procedure 206 may have a result, for example included in the unlabeled data pool 102 or in the embedded pool 104. The result for the selected procedure 206 can be added as a label to the selected procedure 206 during synthesis and testing 208. In other examples in which a result for the selected procedure 206 is not known or accessible, an experiment may be conducted, a simulation may be performed, or a prediction by the artificial intelligence model may be made, to generate a result that can be used to label the selected procedure 206. For example, the artificial intelligence model can be used, or may have previously been used, to generate a prediction of the result for the selected procedure 206, and the prediction of the result can be applied to the selected procedure 206 as the label.

The data flow diagram 200 may proceed to labeled data pool 210, which may be augmented with the labeled data from the selected procedure 206 such as after synthesis and testing 208. For example, the selected procedure 206, with the result as the label from the synthesis and testing 208, can be augmented with or otherwise added to the labeled data pool 210 such that the total number of sets of data included in the labeled data pool 210 increases by one, if only one procedure was selected from the sub-pool; the number of procedures selected from the sub-pool is another hyperparameter—the labeled data associated with the selected procedure(s) 206.

The data flow diagram 200 may proceed to prompt 212, which can be generated based on the labeled data pool 210. In some examples, an MMR operation can be performed on the labeled data pool 210 to select the most relevant data points included in the labeled data pool 210 to use for generating the prompt 212. The prompt 212 may be or include input information configured to be input into the artificial intelligence model to cause the artificial intelligence model to output a particular response, which may be or include a reaction procedure or an inverse design thereof. For example, the prompt 212 may include one or more if-then statements in which a first set of if-then statements define reaction procedures, such as the selected procedure 206, along with their corresponding labeled results. The prompt 212 may additionally include a question or an if-statement regarding randomly perturbed results. For example, the prompt 212 may ask what the reaction procedure for synthesizing a reaction influencer with a yield multiplied by a random, or pseudo-random, normal value. The prompt 212 may also include other supplemental information to influence the artificial intelligence model at inference.

The data flow diagram 200 may proceed to inverse design 214, which may be generated by the artificial intelligence model based on the prompt 212. For example, the computing system can provide the prompt 212 as input to the artificial intelligence model, and the artificial intelligence model can generate the inverse design 214. The inverse design 214 can be or include a reaction procedure that can be used to synthesize and implement a reaction influencer based on a yield or other result indicated by the prompt 212. In some examples, the inverse design 214 can be used to synthesize the reaction influencer, and, in other examples, the inverse design 214 can be provided as input for the computing system to iterate the process, or a process similar thereto, illustrated by the data flow diagram 200. For example, the inverse design 214, if not included in the embedded pool 104, can be used in an MMR process to identify a set of similar reaction procedures to generate a subsequent N-sized set of reaction procedures as a subsequent sub-pool.

### Illustrative System

FIG. 4 is an example of a computing system 400 for identifying procedure specifications for synthesizing reaction influencers in accordance with various embodiments. As illustrated in FIG. 4, the computing system 400 includes a computing device 402, a network 422, a cloud-based server system 424, and a database 426. The computing device 402, the cloud-based server system 424, and the database 426 can be in communication with each other via the network 422, which can be any kind of network, including one or more, public, private, wired, and/or wireless networks, that can facilitate communications among components of the computing system 400.

The computing device 402 includes a processing subsystem 410, one or more storage devices 412, a user interface subsystem 404, and a communication subsystem 406. In some examples, the one or more storage devices 412 may be included in the processing subsystem 410. The computing device 402 can be implemented in various configurations in order to provide various functionality. For example, the computing device 402 can be implemented as a portable electronic or communication device such as a smartphone, a wearable device, a tablet, and the like. The foregoing is not intended to be limiting, and the computing device 402 can be implemented as any kind of electronic device that can be configured to identifying procedure specifications for synthesizing and implementing reaction influencers, using a part of or all of the processing, operations, and/or methods disclosed herein.

The processing subsystem 410 includes one or more processors 414, the one or more storage devices 412, and RAM 416. The one or more processors 414 can read one or more programs from the one or more storage devices 412 and execute them using the RAM 416. Each processor of the one or more processors 414 can be of any type such as a microprocessor, a microcontroller, a graphical processing unit, a digital signal processor, an application-specific integrated circuit (ASIC), a field programmable gate array (FPGA), or any combination thereof. In some implementations, the one or more processors 414 can include a set of cores, a set of arrays, one or more coprocessors, and/or one or more layers of local cache memory. The one or more processors 414 can execute one or more programs stored in the one or more storage devices 412 to perform the processing, operations, and/or methods, including parts thereof, described herein.

Each storage device of the one or more storage devices 412 can be non-volatile and can include any type of memory device that retains stored information when powered off. Some examples of memory can include electrically erasable and programmable read-only memory (EEPROM), flash memory, or any other type of non-volatile memory. At least one storage device of the one or more storage devices 412 can include a non-transitory computer-readable storage medium from which the one or more processors 414 can read instructions. A computer-readable storage medium can include electronic, optical, magnetic, or other storage devices capable of providing the one or more processors 414 with computer-readable instructions or other program code. Some examples of a computer-readable storage medium can include magnetic disks, memory chips, read-only memory (ROM), RAM, an ASIC, a configured processor, optical storage, or any other medium from which a computer processor can read the instructions.

The one or more storage devices 412 can be configured to store information and data acquired, received, and/or generated by the computing device 402. The one or more storage devices 412 can be removable storage devices, non-removable storage devices, a combination thereof, and the like. Some examples of removable storage and non-removable storage devices can include solid-state drives (SSDs), magnetic disk devices such as flexible disk drives (FDDs) and hard disk drives (HDDs), optical disk drives such as compact disk (CD) drives and digital versatile disk (DVD) drives, tape drives, and the like.

The user interface subsystem 404 can include one or more devices that are configured to present images, video, graphics, text, information, data, content, and the like and receive input. Some examples of the devices that can be included in the user interface subsystem 404 can include displays, such as liquid crystal displays, light emitting diode displays, organic light emitting diode displays, touchscreen displays, and the like, audio transducers, such as microphones, speakers, and the like, input/output components such as control members, control panels, switches, buttons, keyboards, mice, and the like.

The communication subsystem 406 can be configured to facilitate communications between the computing device 402 and the network 422 and other systems and devices. For example, the communication subsystem 406 can be configured to facilitate communications between the computing device 402 and the cloud-based server system 424, which can, in some implementations, perform some of the processing functions described herein. For example, the cloud-based server system 424 can include or provide one or more language models 432, such as the artificial intelligence model, a machine-learning model, and the like, that can be used to perform operations described herein. In some implementations, using the communication subsystem 406, the cloud-based server system 424 can be used to relay notifications to and/or store data, for example at the database 426, generated by the computing device 402. In some implementations, to enable communications between the network 422 and/or the cloud-based server system 424, the communication subsystem 406 can include one or more communication devices such as wireless communication modules and chips, wired communication modules and chips, chips for communicating over local area networks, wide area networks, cellular networks, satellite networks, fiber optic networks, and the like, systems on chips, and other circuitry that enables the computing device 402 to send and receive data.

The one or more storage devices 412 can include a discovery module 435 that can be used to perform one or more operations described herein. For example, the discovery module 435 can be executed to identifying procedure specifications for synthesizing reaction influencers using in-context learning and Bayesian optimization according to the techniques described herein. The discovery module 435 may include or may have access to an artificial intelligence model such as the one or more language model 432. Additionally, or alternatively, the discovery module 435 may include one or more sub-modules or services that can perform Bayesian optimization, MMR, and the like.

Although not shown, the computing device 402 can also include other components that can provide the computing device 402 with various functionality. Such other components can include power generating/storing devices, input/output (I/O) components, and the like. The foregoing configurations of the computing device 402 are not intended to be limiting and the computing device 402 can include other subsystems, devices, and components.

### Illustrative Methods

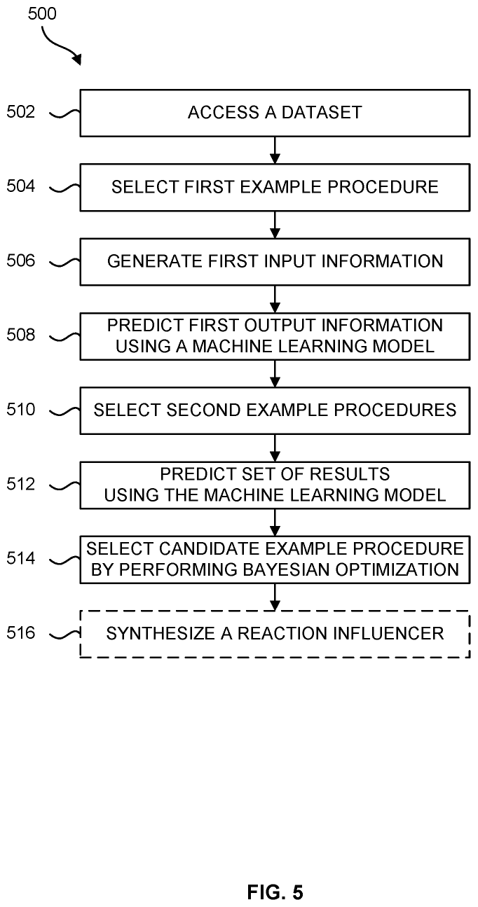

FIG. 5 is a flowchart of a process 500 for generating inverse designs of reaction procedures using in-context learning and Bayesian optimization in accordance with various embodiments. The processing indicated by the flowchart in FIG. 5 may be implemented in software, such as code, instructions, or program, executed by a processing system such as the processing subsystem 410 of the computing device 402. The software may be stored on a non-transitory computer-readable storage medium such as a memory device. The process 500 is intended to be illustrative and non-limiting. For example, although FIG. 5 depicts the various processing steps occurring in a particular sequence or order, in other embodiments, the steps may be performed in some different order or some steps may also be performed at least partially in parallel.

The process 500 begins at block 502 in which a dataset is accessed. In some examples, the computing system 400 can access the dataset, and the dataset can include a set of reaction procedures or other procedures. The set of reaction procedures may include results such as a yield or a solubility of a reaction influencer. In some examples, a first subset of the reaction procedures may include results, and a second subset of the reaction procedures may not include results. Additionally, or alternatively, the set of procedures can be used to synthesize a set of example reaction influencers.

At block 504, a first procedure is selected. The computing system 400 can convert the dataset into a set of vectors, a set of tokens, or the like to allow the computing system 400 to further process the dataset. In some examples, the computing system 400 can use a machine-learning model that includes an embedding layer to embed the set of reaction procedures included in the dataset. The computing system can select, from the embedded set of reaction procedures, the first procedure. In some examples, the computing system selects the first procedure at random or at pseudo-random. Additionally or alternatively, the computing system may select the first procedure from the first subset of reaction procedures to ensure that the first procedure has known results. For example, the selected first procedure may include a procedure specification, which indicates instructions for carrying out the first procedure, and a corresponding label, such as a yield label, that indicates a result, such as a yield or solubility of a corresponding reaction influencer, of the first procedure.

At block 506, first input information is generated. In some examples, the first input information may be or include a prompt that can be provided to an artificial intelligence or machine-learning model to cause the artificial intelligence or machine-learning model to generate a response. The first input information can be generated based at least in part on the first procedure. For example, the first input information can include a natural language description of the first procedure. Additionally or alternatively, the first input information can include a first query that can include the yield label and a normal random variable. In some examples, the yield label, or a value indicated thereby, can be multiplied by the normal random variable within the first query.

At block 508, first output information is predicted using a machine-learning model. The machine-learning model may be or include a pre-trained generative artificial intelligence model such as any suitable version of a GPT model provided by OpenAI. The machine-learning model can include any other suitable type or version of a pre-trained machine-learning model. In some examples, the first output information can be generated for, or based at least in part on, the first query. The first output information can include a natural language description of a reaction influencer and a procedure, or procedure specification, for synthesizing and testing the reaction influencer.

At block 510, second procedures are selected. In some examples, more than one procedure can be selected as the second procedures. The second procedures can include two procedures, three procedures, four procedures, five procedures, ten procedures, 15 procedures, or more than 15 procedures. In some examples, the second procedures can be selected based at least in part on an MMR operation performed by the computing system 400 on the procedure provided by the output information. Optionally, and in some examples, such as examples in which the procedure provided by the output information is included in the embedded reaction procedures, the process 500 may proceed to block 516.

At block 512, a set of results for the second procedures is predicted by the machine-learning model. The computing system 400 can provide the second procedures to the machine-learning model in a subsequent prompt or subsequent input information, and the machine-learning model can provide subsequent output information that includes predictions of results, such as yields of corresponding reaction influencers, for the second procedures. Additionally or alternatively, the machine-learning model may generate and include in the subsequent output information a set of certainty factors for the set of results. Each certainty factor of the set of certainty factors can indicate a confidence or uncertainty of a corresponding result of the set of results.

At block 514, a candidate procedure is selected by performing Bayesian optimization. The computing system 400 can provide the subsequent output information to a Bayesian optimization service or model, which can perform Bayesian optimization to select the candidate procedure. The Bayesian optimization can use the set of results and the set of certainty factors to identify the input values that may optimize, such as maximize, an objective function. In some examples, the objective function may be or include an acquisition function like an expected improvement function or an upper confidence bound function. An output of the Bayesian optimization can include the candidate procedure indicated as the optimal procedure to test next for synthesizing the reaction influencer.

At block 516, the reaction influencer is synthesized and tested. In some examples, the candidate procedure can be used to synthesize the reaction influencer to perform additional iterations of the process 500. In other examples, the candidate procedure can be used to synthesize the reaction influencer to use the reaction influencer in a separate chemical reaction. Additionally or alternatively, the reaction influencer can be synthesized using the procedure, such as an inverse design of a reaction procedure, indicated by the first output information.

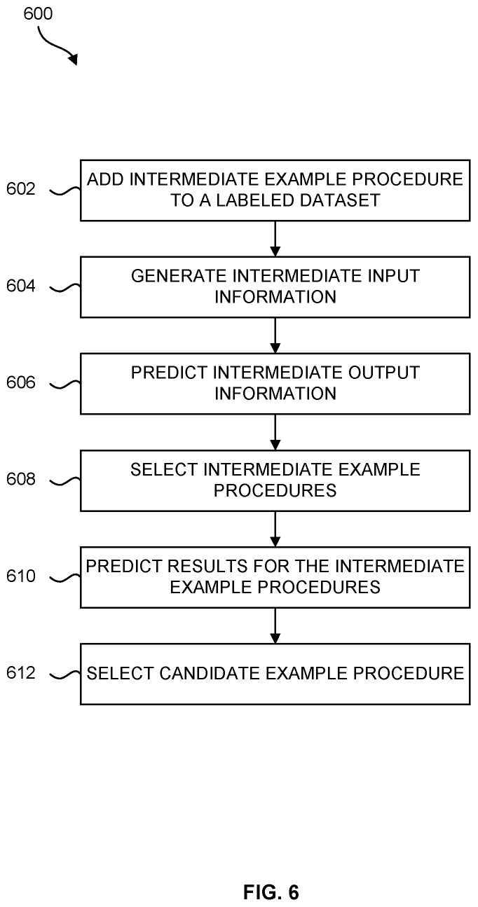

FIG. 6 is a flowchart of a process 600 for performing Bayesian optimization for identifying a candidate procedure in accordance with various embodiments. The processing indicated by the flowchart in FIG. 6 may be implemented in software, such as code, instructions, or program, executed by a processing system such as the processing subsystem 410 of the computing device 402. The software may be stored on a non-transitory computer-readable storage medium such as a memory device. The process 600 is intended to be illustrative and non-limiting. For example, although FIG. 6 depicts the various processing steps occurring in a particular sequence or order, in other embodiments, the steps may be performed in some different order or some steps may also be performed at least partially in parallel.

The process 600 begins at block 602 in which an intermediate procedure is added to a labeled dataset. In some examples, the labeled dataset may be similar or identical to the labeled data pool 110, the labeled data pool 210, the labeled data pool 302, or any combination thereof. The intermediate procedure may have previously been selected from a previously run Bayesian optimization operation. For example, the intermediate procedure can be identified from a set of procedures using an acquisition function in the previously run Bayesian optimization operation.

At block 604, intermediate input information is generated. The computing system 400 may generate the intermediate input information for input into the machine-learning model. In some examples, the intermediate input information may be based at least in part on one or more intermediate procedures included in the labeled dataset. For example, the computing system 400 can apply an MMR operation on the procedures included in the labeled dataset to select a subset of the procedures as the intermediate procedures that can be used to generate the intermediate input information. In some examples, a reference procedure used with respect to the MMR operation can be or include a best or augmented maximum value in the labeled pool as reference. The augmented maximum value may involve being augmented with, or otherwise adjusted by, a normal random variable.

At block 606, intermediate output information is predicted. In some examples, the machine-learning model can be used to generate the intermediate output information based at least in part on the intermediate input information. The computing system 400 can provide the intermediate procedures to the machine-learning model via the intermediate input information, and the machine-learning model can provide the intermediate output information that includes an inverse design of a reaction procedure.

At block 608, a set of intermediate procedures is selected. The computing system 400 can select the set of intermediate procedures based at least in part on the intermediate output information, or the inverse design included therein. In some examples, more than one procedure can be selected as the set of intermediate procedures. The set of intermediate procedures can include any number of procedures such as two procedures, two-three procedures, three-four procedures, four-five procedures, five-ten procedures, ten-15 procedures, more than 15 procedures, and so on. In some examples, the set of intermediate procedures can be selected based at least in part on an MMR operation performed by the computing system 400 on the inverse design of the reaction procedure provided by the intermediate output information.

At block 610, results are predicted for the set of intermediate procedures. The computing system 400 can use the machine-learning model to generate predicted results for each intermediate procedure of the set of intermediate procedures. For example, the computing system 400 can provide the set of intermediate procedures to the machine-learning model in a subsequent prompt or subsequent input information, and the machine-learning model can provide subsequent output information that includes predictions of results, such as yields of corresponding reaction influencers, for the set of intermediate procedures. Additionally or alternatively, the machine-learning model may generate and include in the subsequent output information a set of certainty factors for the set of results. Each certainty factor of the set of certainty factors can indicate a confidence or uncertainty of a corresponding result of the set of results.

At block 612, a candidate procedure is selected. The computing system 400 can use Bayesian optimization, or any other suitable optimization procedure or combination of optimization procedures, to identify and/or select the candidate procedure. For example, the computing system 400 can use the set of results and the set of certainty factors to optimize an objection function. In some examples, the objective function may be or include the acquisition function such as an expected improvement function, or an upper confidence bound function. An output of the Bayesian optimization can include the candidate procedure indicated as the optimal procedure to test next for synthesizing the reaction influencer.

The operations illustrated and described with respect to the process 600 may be iterative. For example, blocks 602-612 may be configured to be performed multiple, such as a predetermined number of, times to reach a desired procedure or for other suitable purposes.

Although specific embodiments have been described, various modifications, alterations, alternative constructions, and equivalents are also encompassed within the scope of the disclosure. Embodiments are not restricted to operation within certain specific data processing environments but are free to operate within a plurality of data processing environments. Additionally, although embodiments have been described using a particular series of transactions and steps, it should be apparent to those skilled in the art that the scope of the present disclosure is not limited to the described series of transactions and steps. Various features and aspects of the above-described embodiments may be used individually or jointly.

Further, while embodiments have been described using a particular combination of hardware and software, it should be recognized that other combinations of hardware and software are also within the scope of the present disclosure. Embodiments may be implemented only in hardware, or only in software, or using combinations thereof. The various processes described herein can be implemented on the same processor or different processors in any combination. Accordingly, where components or services are described as being configured to perform certain operations, such configuration can be accomplished, e.g., by designing electronic circuits to perform the operation, by programming programmable electronic circuits (such as microprocessors) to perform the operation, or any combination thereof. Processes can communicate using a variety of techniques including but not limited to conventional techniques for inter process communication, and different pairs of processes may use different techniques, or the same pair of processes may use different techniques at different times.

The specification and drawings are, accordingly, to be regarded in an illustrative rather than a restrictive sense. It will, however, be evident that additions, subtractions, deletions, and other modifications and changes may be made thereunto without departing from the broader spirit and scope as set forth in the claims. Thus, although specific disclosure embodiments have been described, these are not intended to be limiting. Various modifications and equivalents are within the scope of the following claims.

The use of the terms “a” and “an” and “the” and similar referents in the context of describing the disclosed embodiments (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments and does not pose a limitation on the scope of the disclosure unless otherwise claimed. No language in the specification should be construed as indicating any non-claimed element as essential to the practice of the disclosure.

As used herein, when an action is “based on” something, this means the action is based at least in part on at least a part of the something. As used herein, the terms “substantially,” “approximately” and “about” are defined as being largely but not necessarily wholly what is specified (and include wholly what is specified) as understood by one of ordinary skill in the art. In any disclosed embodiment, the term “substantially,” “approximately,” or “about” may be substituted with “within [a percentage] of” what is specified, where the percentage includes 0.1, 1, 5, and 10 percent.

Disjunctive language such as the phrase “at least one of X, Y, or Z,” unless specifically stated otherwise, is intended to be understood within the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Preferred embodiments of this disclosure are described herein, including the best mode known for carrying out the disclosure. Variations of those preferred embodiments may become apparent to those of ordinary skill in the art upon reading the foregoing description. Those of ordinary skill should be able to employ such variations as appropriate and the disclosure may be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein.

All references, including publications, patent applications, and patents, cited herein are hereby incorporated by reference to the same extent as if each reference were individually and specifically indicated to be incorporated by reference and were set forth in its entirety herein.

In the foregoing specification, aspects of the disclosure are described with reference to specific embodiments thereof, but those skilled in the art will recognize that the disclosure is not limited thereto. Various features and aspects of the above-described disclosure may be used individually or jointly. Further, embodiments can be utilized in any number of environments and applications beyond those described herein without departing from the broader spirit and scope of the specification. The specification and drawings are, accordingly, to be regarded as illustrative rather than restrictive.

1. A computer-implemented method comprising:
accessing a dataset comprising a set of procedures for synthesizing a plurality of reaction influencers;
selecting a first procedure from a subset of procedures of the set of procedures, the first procedure comprising a procedure specification and a corresponding label;
generating first input information for the first procedure, wherein the first input information comprises a natural language description of the first procedure and a first query, wherein the first query comprises the corresponding label adjusted by a normal random variable;
predicting, based at least in part on the first input information and using a machine learning model, first output information for the first query, wherein the first output information comprises a natural language description of a reaction influencer and a procedure for synthesizing and testing the reaction influencer;
selecting a plurality of second procedures from the set of procedures;
predicting, using the machine learning model based on previously labeled procedures, a set of results for the plurality of second procedures, wherein the set of results comprises a plurality of certainty factors for the set of results; and
selecting a candidate procedure from the set of procedures by performing Bayesian optimization on the plurality of second procedures using the plurality of certainty factors in an acquisition function of the Bayesian optimization.

2. The method of claim 1, wherein each procedure of the set of procedures comprises information identifying an reaction influencer of the plurality of reaction influencers and information identifying procedure conditions for synthesizing and testing the reaction influencer, and wherein each procedure of the subset of procedures comprises the information identifying the reaction influencer, the information describing the procedure conditions, and information identifying a label comprising a yield of a reaction influenced by the reaction influencer.

3. The method of claim 1, wherein the plurality of second procedures is selected from the set of procedures using a maximal marginal relevance function.

4. The method of claim 1, wherein performing Bayesian optimization on the plurality of second procedures comprises performing the following steps:
(i) adding an intermediate procedure to a labeled dataset, the intermediate procedure identified from a plurality of procedures using the acquisition function;
(ii) generating intermediate input information for the machine learning model based one or more intermediate procedures in the labeled dataset;
(iii) predicting, using the machine learning model, intermediate output information for the intermediate input information;
(iv) selecting a plurality of intermediate procedures from the set of procedures based on the intermediate output information;
(v) predicting, using the machine learning model, results for the plurality of intermediate procedures;
(vi) selecting, using the acquisition function, the candidate procedure from the plurality of intermediate procedures; and
(vii) repeating steps (i)-(vi) a predetermined number of times.

5. The method of claim 4, wherein generating the intermediate input information for the machine learning model based on the one or more intermediate procedures in the labeled dataset comprises selecting the one or more intermediate procedures from the labeled dataset using a maximal marginal relevance function.

6. The method of claim 1, wherein the first input information further comprises a natural language description of a third procedure, the third procedure selected from a dataset of labeled procedures.

7. The method of claim 1, wherein each respective result of the set of results comprises a yield for a reaction influencer associated with a second procedure of the plurality of second procedures and a certainty factor of the plurality of certainty factors.

8. A system comprising:
one or more processors; and
one or more computer-readable media storing instructions which, when executed by the one or more processors, cause the system to perform operations comprising:
accessing a dataset comprising set of procedures for synthesizing a plurality of reaction influencers;
selecting a first procedure from a subset of procedures of the set of procedures, the first procedure comprising a procedure specification and a corresponding label;
generating first input information for the first procedure, wherein the first input information comprises a natural language description of the first procedure and a first query, wherein the first query comprises the corresponding label adjusted by a normal random variable;
predicting, based at least in part on the first input information and using a machine learning model, first output information for the first query, wherein the first output information comprises a natural language description of a reaction influencer and a procedure for synthesizing the reaction influencer;
selecting a plurality of second procedures from the set of procedures;
predicting, using the machine learning model, a set of results for the plurality of second procedures, wherein the set of results comprises a plurality of certainty factors for the set of results; and
selecting a candidate procedure from the set of procedures by performing Bayesian optimization on the plurality of second procedures using the plurality of certainty factors in an acquisition function of the Bayesian optimization.

9. The system of claim 8, wherein each procedure of the set of procedures comprises information identifying an reaction influencer of the plurality of reaction influencers and information identifying procedure conditions for synthesizing the reaction influencer, and wherein each procedure of the subset of procedures comprises the information identifying the reaction influencer, the information describing the procedure conditions, and information identifying a label comprising a yield of a reaction influenced by the reaction influencer.

10. The system of claim 8, wherein the plurality of second procedures is selected from the set of procedures using a maximal marginal relevance function.

11. The system of claim 8, wherein performing Bayesian optimization on the plurality of second procedures comprises performing the following steps:
(i) adding an intermediate procedure to a labeled dataset, the intermediate procedure identified from a plurality of procedures using the acquisition function;
(ii) generating intermediate input information for the machine learning model based one or more intermediate procedures in the labeled dataset;
(iii) predicting, using the machine learning model, intermediate output information for the intermediate input information;
(iv) selecting a plurality of intermediate procedures from the set of procedures based on the intermediate output information;
(v) predicting, using the machine learning model, results for the plurality of intermediate procedures;
(vi) selecting, using the acquisition function, the candidate procedure from the plurality of intermediate procedures; and
(vii) repeating steps (i)-(vi) a predetermined number of times.

12. The system of claim 11, wherein generating the intermediate input information for the machine learning model based on the one or more intermediate procedures in the labeled dataset comprises selecting the one or more intermediate procedures from the labeled dataset using a maximal marginal relevance function.

13. The system of claim 8, wherein the first input information further comprises a natural language description of a third procedure, the third procedure selected from a dataset of labeled procedures.

14. The system of claim 9, wherein each respective result of the set of results comprises a yield for a reaction influencer associated with a second procedure of the plurality of second procedures and a certainty factor of the plurality of certainty factors.

15. One or more non-transitory computer-readable media storing instructions which, when executed by one or more processors, cause a system to perform operations comprising:
accessing a dataset comprising set of procedures for synthesizing a plurality of reaction influencers;
selecting a first procedure from a subset of procedures of the set of procedures, the first procedure comprising a procedure specification and a corresponding label;
generating first input information for the first procedure, wherein the first input information comprises a natural language description of the first procedure and a first query, wherein the first query comprises the corresponding label adjusted by a normal random variable;
predicting, based at least in part on the first input information and using a machine learning model, first output information for the first query, wherein the first output information comprises a natural language description of a reaction influencer and a procedure for synthesizing the reaction influencer;
selecting a plurality of second procedures from the set of procedures;
predicting, using the machine learning model, a set of results for the plurality of second procedures, wherein the set of results comprises a plurality of certainty factors for the set of results; and
selecting a candidate procedure from the set of procedures by performing Bayesian optimization on the plurality of second procedures using the plurality of certainty factors in an acquisition function of the Bayesian optimization.

16. The one or more non-transitory computer-readable media of claim 15, wherein each procedure of the set of procedures comprises information identifying an reaction influencer of the plurality of reaction influencers and information identifying procedure conditions for synthesizing the reaction influencer, and wherein each procedure of the subset of procedures comprises the information identifying the reaction influencer, the information describing the procedure conditions, and information identifying a label comprising a yield of a reaction influenced by the reaction influencer.

17. The one or more non-transitory computer-readable media of claim 15, wherein the plurality of second procedures is selected from the set of procedures using a maximal marginal relevance function.

18. The one or more non-transitory computer-readable media of claim 15, wherein performing Bayesian optimization on the plurality of second procedures comprises performing the following steps:
(i) adding an intermediate procedure to a labeled dataset, the intermediate procedure identified from a plurality of procedures using the acquisition function;
(ii) generating intermediate input information for the machine learning model based one or more intermediate procedures in the labeled dataset;
(iii) predicting, using the machine learning model, intermediate output information for the intermediate input information;
(iv) selecting a plurality of intermediate procedures from the set of procedures based on the intermediate output information;
(v) predicting, using the machine learning model, results for the plurality of intermediate procedures;
(vi) selecting, using the acquisition function, the candidate procedure from the plurality of intermediate procedures; and
(vii) repeating steps (i)-(vi) a predetermined number of times.

19. The one or more non-transitory computer-readable media of claim 18, wherein generating the intermediate input information for the machine learning model based on the one or more intermediate procedures in the labeled dataset comprises selecting the one or more intermediate procedures from the labeled dataset using a maximal marginal relevance function.

20. The one or more non-transitory computer-readable media of claim 15, wherein the first input information further comprises a natural language description of a third procedure, the third procedure selected from a dataset of labeled procedures.
